(19) United States
(12) Patent Application Publication
(10) Pub. No.: US 2017/0028080 A1
(43) Pub. Date: Feb. 2, 2017

(54) Targeted Drug Conjugates

(71) Applicant: Philochem AG, Otelfingen (CH)

(72) Inventors: Giulio Casi, Opfikon (CH); Francesca Pretto, Zurich (CH)

(21) Appl. No.: 15/227,796

(22) Filed: Aug. 3, 2016

Related U.S. Application Data
(63) Continuation of application No. PCT/EP2015/052205, filed on Feb. 3, 2015.

(30) Foreign Application Priority Data
Feb. 3, 2014 (GB) 1401818.8
Apr. 29, 2014 (GB) 1407533.7
Nov. 10, 2014 (GB) 1419996.2
Dec. 16, 2014 (GB) 1422399.4

(51) Int. Cl. A61K 47/48; C07K 16/30; C07K 16/18

(52) U.S. Cl. CPC A61K 47/48569; C07K 16/18; A61K 47/48384; C07K 16/3038; C07K 2317/35; C07K 2317/622; C07K 2317/55

(57) ABSTRACT

A targeted therapeutic agent comprising a compound of formula:

B-L-D

wherein: B is a non-internalizing binding moiety specific for a cancer associated protein; D is a cytotoxic drug moiety; and L is a linker group that undergoes cleavage in vivo for releasing said drug moiety in an active form. The binding moiety is a ligand for the cancer associated protein whereby drawbacks associated with the use of internalizing ligands are avoided.

### CROSS-REFERENCE TO RELATED APPLICATIONS

This application is a continuation of PCT application PCT/EP2015/052205 filed Feb. 3, 2015, which claims benefit of GB 1422399.4 filed Dec. 16, 2014, GB 1419996.2 filed Nov. 10, 2014, GB 1407533.7 filed Apr. 29, 2014, and GB 1401818.8 filed Feb. 3, 2014. The contents of the above patent applications are incorporated by reference herein in their entirety.

### FIELD OF THE INVENTION

The present invention relates to the field of targeted drug conjugates for the treatment of disease. In particular, the invention relates to non-internalizing drug conjugates formed of a targeting ligand conjugated to a drug by a cleavable linker for delivery of the drug to targeted tissues or cells. In one embodiment, the present invention relates to the application of the targeted drug conjugates for the delivery of drugs that can kill or inhibit tumour cells.

### BACKGROUND

The use of cytotoxic agents is at the basis of the treatment of cancer and other pathological conditions. Ideally cytotoxic agents should accumulate at site of disease, sparing normal tissues. In reality this does not happen. Many anticancer drugs do not preferentially accumulate in solid tumors. Indeed, it has been demonstrated in tumor-bearing mice that only a minimal portion of the injected drug reaches the neoplastic mass in comparison to the amount of cytotoxic agent that reaches healthy organs. More importantly, emerging Positron Emission Tomography (PET) studies, performed with radiolabeled cytotoxic drugs (e.g., ¹¹C-docetaxel) have unequivocally shown that these toxic agents do not preferentially accumulate on neoplastic lesions, but rather target other structures in the body (e.g. clearance-associated organs).

The targeted delivery of highly potent cytotoxic agents into diseased tissues is therefore desirable for the treatment of cancer and other serious conditions. By attaching a therapeutic effector through a cleavable linker to a ligand specific to a marker of disease, the effector preferentially accumulates and acts at the intended site of action, thus increasing the effectively applied dose while reducing side effects. To date, monoclonal antibodies capable of selective internalization into the target tumor cells have been considered as the ligands of choice and, indeed, research in the field of antibody-drug conjugates (ADCs) has led to the recent approval of two ADCs for applications in oncology: brentuximab vedotin and trastuzumab emtansine.

However, antibodies are large macromolecules and thus often have difficulties penetrating deeply into solid tumors. In addition, they can be immunogenic and typically long circulation times can lead to premature drug release and undesired side effects. Moreover, the production of ADCs is expensive, reflecting the need for clinical-grade manufacturing of antibodies, drugs and the resulting conjugates.

The use of smaller ligands as delivery vehicles such as peptides or small drug-like molecules capable of selective internalization into tumor cells could potentially overcome some of the abovementioned problems. Their reduced size should aid tissue penetration, they should be non-immunogenic and amenable to classic organic synthesis thus reducing manufacturing costs. The favorable properties of drug conjugates using folic acid or ligands against prostate-specific membrane antigen (PSMA) as delivery vehicles have been demonstrated and a folate conjugate has recently entered Phase III clinical studies. However, only a few such conjugates have been successfully identified.

Existing cytotoxic drug conjugates are activated inside the cells after they have been internalized into the cells by active endocytosis, such as by receptor/antigen mediated cytosis. This has the drawback that only a very small proportion of the drug is released inside the cells of interest, and a larger proportion may accumulate in normal tissues and cause undesired side effects. Moreover, the cytotoxic drug released inside the cell may give only very local toxicity, and in particular may not kill neighboring cells that have not internalized the drug, for example because they lack the relevant cell surface antigen.

The present inventors have found drug conjugates, including small molecule drug conjugates, that target proteins that are expressed on the endothelial cells or in the surrounding stroma of tumours (i.e., not on tumor cells), and which do not internalize into tumor cells, but rather set free their toxic payload in the extracellular milieu.

### SUMMARY OF THE INVENTION

The present invention is based on the discovery that cytotoxic drug conjugates that target the tumor microenvironment and that do not rely on internalization can be curative in mouse models of cancer.

The strong antitumor activity reported in the inventive conjugates based on non-internalizing ligands (antibody or small molecule) were surprising as it is generally believed that ligands capable of selective internalization into the tumor cells are needed for efficient intracellular drug delivery. Indeed, it has been claimed that targeting an ADC to a noninternalizing target antigen with the expectation that extracellulary released drug will diffuse into the target cell is not a recipe for a successful ADC.

According to the first aspect of the invention, therefore, there is provided a targeted therapeutic agent comprising a compound of formula:

B-L-D

wherein:

B is a binding moiety specific for a cancer associated antigen;

D is a cytotoxic drug moiety; and

L is a linker group that undergoes cleavage in vivo for releasing said drug moiety in an active form.

Preferably, the binding moiety B is a non-internalizing binding moiety. Likewise, the drug conjugates of the invention are preferably non-internalizing. A “non-internalizing” moiety has the property of reacting in physiological conditions (at 37° C. and pH 7) in vivo or in vitro, with binding partners on the cell surface (e.g. cell surface antigens) or in the extracellular matrix without being internalized in the cells by a process of active endocytosis (such as receptor/antigen mediated endocytosis). It is possible that some of the non-internalizing specific binding moiety could be taken up intracellularly by fluid phase endocytosis. However, the amount of fluid phase endocytosis will depend linearly on the extracellular binding moiety concentration and temperature and can therefore be distinguished from mediated endocytosis in order to distinguish non-internalizing binding moieties and conjugates according to the present invention.

Suitably, the binding moiety is a low molecular weight binding moiety, whereby the compound of Formula (I) is a low molecular weight drug conjugate, also referred to as a small molecule drug conjugate (SMDC). Suitably, the SMDC has a molecular weight less than about 10,000, more suitably less than about 5000, and most suitably less than about 2000. In contrast to antibodies, small molecules can diffuse out of blood vessels in a matter of seconds. The distribution is not restricted to perivascular space, but involves also deep penetration into tissues. This results in faster, deeper and more efficient drug targeting by the agents of the invention.

The target antigen is suitably a protein that is expressed on the endothelial cells or in the surrounding stroma of a tumor, or that is released following tumor cell death.

Thus, in an aspect of the present invention there is provided a targeted therapeutic agent comprising a compound of formula:

B-L-D (I),

wherein:

B is a low molecular weight binding moiety specific for a protein that is expressed on the endothelial cells or in the surrounding stroma of tumours;

D is a drug moiety; and

L is a linker group that undergoes cleavage in vivo for releasing said drug moiety in an active form at said disease site.

In another aspect, the present invention provides a targeted therapeutic agent in accordance with the invention, for use in the treatment of a neoplastic disease, preferably for the treatment of a solid tumor, more preferably for the treatment of renal cell carcinoma.

In another aspect, the present invention provides a pharmaceutical composition comprising a targeted therapeutic agent according to the invention.

In another aspect, the present invention provides a product comprising a compound of Formula (I) as defined herein and a cleavage agent for cleaving said cleavable linker L, as a combined preparation for sequential administration in the treatment of cancer.

In another aspect, the present invention provides a method of treating a neoplastic disease, preferably a solid tumor such as renal cell carcinoma, comprising administering an effective amount of a pharmaceutical composition according to the present invention to a patient in need thereof. In embodiments, the administration of said pharmaceutical composition is followed after a suitable interval of time by administration of a cleavage agent for cleaving said cleavable linker L.

Any feature described herein as suitable, optional, or preferred in relation to any one aspect of the invention may likewise be suitable, optional or preferred in relation to any other aspect of the invention.

### BRIEF DESCRIPTION OF THE DRAWINGS

FIG. 1 shows structures of MMP inhibitors suitable for use in the binding moieties of the conjugates of the present invention;

FIG. 2 shows structures of FAP inhibitors suitable for use in the binding moieties of the conjugates of the present invention;

FIG. 3 shows general structures of cleavable moieties suitable for use in the linker moieties of the present invention;

FIG. 4 shows structures of three drug conjugates according to the invention;

FIG. 5 shows data observed for mouse tumor size versus time for Auristatin (MMAE) conjugated with F8 Antibody binding moieties and Cathepsin B-Cleavable Peptide Linker, together with comparative data for reference compounds and controls;

FIG. 6 shows toxicity data observed for the examples and comparative examples of FIG. 5;

FIG. 7 shows data observed for mouse tumor size versus time for Auristatin (MMAE) conjugated with F16 Antibody binding moieties and Cathepsin B-Cleavable Peptide Linker, together with comparative data for reference compounds and controls; and

FIG. 8 shows toxicity data observed for the examples and comparative examples of FIG. 7.

### DETAILED DESCRIPTION OF THE INVENTION

Definitions

Antibody. The term “antibody” is used in its broadest sense and covers monoclonal antibodies, polyclonal antibodies, dimers, multimers, multispecific antibodies (eg. bispecific antibodies), veneered antibodies, antibody fragments and small immune proteins (SIPs) (see Int. J. Cancer (2002) 102, 75-85). An antibody is a protein generated by the immune system that is capable of recognizing and binding to a specific antigen. A target antigen generally has numerous binding sites, also called epitopes, recognized by CDRs on multiple antibodies. Each antibody that specifically binds to a different epitope has a different structure. Thus, one antigen may have more than one corresponding antibody. An antibody includes a full-length immunoglobulin molecule or an immunologically active portion of a full-length immunoglobulin molecule, ie. a molecule that contains an antigen binding site that immunospecifically binds an antigen of a target of interest or part thereof. The antibodies may be of any type—such as IgG, IgE, IgM, IgD, and IgA)—any class—such as IgG1, IgG2, IgG3, IgG4, IgA1 and IgA2—or subclass thereof. The antibody may be or may be derived from murine, human, rabbit or from other species.

Antibody fragments. The term “antibody fragment” refers to a portion of a full length antibody, generally the antigen binding or variable region thereof. Examples of antibody fragments include, but are not limited to, Fab, Fab′, F(ab′)₂, and Fv fragments; diabodies; linear antibodies; single domain antibodies, including dAbs, camelid V_{HH} antibodies and the IgNAR antibodies of cartilaginous fish. Antibodies and their fragments may be replaced by binding molecules based on alternative non-immunoglobulin scaffolds, peptide aptamers, nucleic acid aptamers, structured polypeptides comprising polypeptide loops subtended on a non-peptide backbone, natural receptors or domains thereof.

Linker. A “linker” means a chemical moiety comprising a covalent bond or a chain of atoms that covalently attaches a protein to a drug moiety.

Derivative. A derivative includes the chemical modification of a compound. Examples of such modifications include the replacement of a hydrogen by a halo group, an alkyl group, an acyl group or an amino group and the like. The modification may increase or decrease one or more hydrogen bonding interactions, charge interactions, hydrophobic interactions, van der Waals interactions and/or dipole interactions.

Analog. This term encompasses any enantiomers, racemates and stereoisomers, as well as all pharmaceutically acceptable salts and hydrates of such compounds.

Unless otherwise stated, the following definitions apply to chemical terms used in connection of compounds of the invention and compositions containing such compounds.

Alkyl refers to a branched or unbranched saturated hydrocarbyl radical. Suitably, the alkyl group comprises from about 3 to about 30 carbon atoms, for example from about 5 to about 25 carbon atoms.

Alkenyl refers to a branched or unbranched hydrocarbyl radical containing one or more carbon-carbon double bonds. Suitably, the alkenyl group comprises from about 3 to about 30 carbon atoms, for example from about 5 to about 25 carbon atoms.

Alkynyl refers to a branched or unbranched hydrocarbyl radical containing one or more carbon-carbon triple bonds. Suitably, the alkynyl group comprises from about 3 to about 30 carbon atoms, for example from about 5 to about 25 carbon atoms.

Halogen refers to fluorine, chlorine, bromine or iodine, preferably fluorine or chlorine.

Cycloalkyl refers to an alicyclic moiety, suitably having 3, 4, 5, 6, 7 or 8 carbon atoms. The group may be a bridged or polycyclic ring system. More often cycloalkyl groups are monocyclic. This term includes reference to groups such as cyclopropyl, cyclobutyl, cyclopentyl, cyclohexyl, norbornyl, bicyclo[2.2.2]octyl and the like.

Aryl refers to an aromatic ring system comprising 6, 7, 8, 9, 10, 11, 12, 13, 14, 15 or 16 ring carbon atoms. Aryl may be a polycyclic ring system, having two or more rings, at least one of which is aromatic. This term includes reference to groups such as phenyl, naphthyl, fluorenyl, azulenyl, indenyl, anthryl and the like.

The prefix (hetero) herein signifies that one or more of the carbon atoms of the group may be substituted by nitrogen, oxygen, phosphorus, silicon or sulfur. Heteroalkyl groups include for example, alkyloxy groups and alkythio groups. Heterocycloalkyl or heteroaryl groups herein may have from 3, 4, 5, 6, 7, 8, 9, 10, 11, 12, 13, 14, 15 or 16 ring atoms, at least one of which is selected from nitrogen, oxygen, phosphorus, silicon and sulfur. In particular, a 3- to 10-membered ring or ring system and more particularly a 5- or 6-membered ring, which may be saturated or unsaturated. For example, selected from oxiranyl, azirinyl, 1,2-oxathiolanyl, imidazolyl, thienyl, furyl, tetrahydrofuryl, pyranyl, thiopyranyl, thianthrenyl, isobenzofuranyl, benzofuranyl, chromenyl, 2H-pyrrolyl, pyrrolyl, pyrrolinyl, pyrrolidinyl, imidazolyl, imidazolidinyl, benzimidazolyl, pyrazolyl, pyrazinyl, pyrazolidinyl, thiazolyl, isothiazolyl, dithiazolyl, oxazolyl, isoxazolyl, pyridyl, pyrazinyl, pyrimidinyl, piperidyl, piperazinyl, pyridazinyl, morpholinyl, thiomorpholinyl, especially thiomorpholino, indolizinyl, 1,3-Dioxo-1,3-dihydro-isoindolyl, 3H-indolyl, indolyl, benzimidazolyl, cumaryl, indazolyl, triazolyl, tetrazolyl, purinyl, 4H-quinolizinyl, isoquinolyl, quinolyl, tetrahydroquinolyl, tetrahydroisoquinolyl, decahydroquinolyl, octahydroisoquinolyl, benzofuranyl, dibenzofuranyl, benzothiophenyl, dibenzothiophenyl, phthalazinyl, naphthyridinyl, quinoxalyl, quinazolinyl, quinazolinyl, cinnolinyl, pteridinyl, carbazolyl, [beta]-carbolinyl, phenanthridinyl, acridinyl, perimidinyl, phenanthrolinyl, furazanyl, phenazinyl, phenothiazinyl, phenoxazinyl, chromenyl, isochromanyl, chromanyl, 3,4-dihydro-2H-isoquinolin-1-one, 3,4-dihydro-2H-isoquinolinyl, and the like.

“Substituted” signifies that one or more, especially up to 5, more especially 1, 2 or 3, of the hydrogen atoms in said moiety are replaced independently of each other by the corresponding number of substituents. The term “optionally substituted” as used herein includes substituted or unsubstituted. It will, of course, be understood that substituents are only at positions where they are chemically possible, the person skilled in the art being able to decide (either experimentally or theoretically) without inappropriate effort whether a particular substitution is possible. For example, amino or hydroxy groups with free hydrogen may be unstable if bound to carbon atoms with unsaturated (e.g. olefinic) bonds. Additionally, it will of course be understood that the substituents described herein may themselves be substituted by any substituent, subject to the aforementioned restriction to appropriate substitutions as recognised by the skilled person.

Substituents may suitably include halogen atoms and halomethyl groups such as CF₃ and CCl₃; oxygen containing groups such as oxo, hydroxy, carboxy, carboxyalkyl, alkoxy, alkoyl, alkoyloxy, aryloxy, aryloyl and aryloyloxy; nitrogen containing groups such as amino, alkylamino, dialkylamino, cyano, azide and nitro; sulfur containing groups such as thiol, alkylthiol, sulfonyl and sulfoxide; heterocyclic groups which may themselves be substituted; alkyl groups, which may themselves be substituted; and aryl groups, which may themselves be substituted, such as phenyl and substituted phenyl. Alkyl includes substituted and unsubstituted benzyl.

Where two or more moieties are described as being “each independently” selected from a list of atoms or groups, this means that the moieties may be the same or different. The identity of each moiety is therefore independent of the identities of the one or more other moieties.

Target

The present invention targets primarily proteins that are expressed on the endothelial cells or in the surrounding stroma of tumours. Suitably, the target proteins are not expressed or over-expressed on tumor cells. Suitably, the target protein is an extracellular matrix (ECM) protein. In embodiments, the invention targets such proteins that are over-expressed at a tumor site, or that are released following tumor cell death. In other embodiments, the invention specifically targets variants of such proteins having modified structures that are expressed specifically at tumor sites.

It has long been known that the endothelium and surrounding stroma in tumours differs from that in normal tissue, but only recently have these differences begun to be characterized at the molecular level. Proteins expressed specifically on the tumour vasculature but not on the vasculature of normal tissues can not only be used for anti-tumour targeting but also for diagnostic (e.g. imaging) purposes. The specific accumulation at the tumour vasculature actively reduces the toxic side effects that are typically associated with the anti-tumour compounds at other locations in the normal tissue and, consequently, allows for the reduction of the concentration of the toxic agents.

In one embodiment, the drug conjugates of the present invention localise at vascular tissue or at a vascular cell in vivo. In embodiments, the drug conjugate localises at the sub-endothelial extracellular matrix in vivo. Suitably, the compound localises at a vascular tumour in vivo.

Preferably, the drug conjugate does not internalise into a targeted tissue or cell in vivo. Thus, the drug conjugates suitably do not substantially internalize into tumor cells, but rather set free their toxic payload predominantly in the extracellular milieu. The use of non-internalizing compounds provides advantages. For example, internalization efficiency is difficult to measure in vivo, thus remaining a “black box” for drug development. Moreover, it is difficult to ensure that all diseased cells are targeted by internalizing compounds, especially those cells which are further away from blood vessels. In contrast, the cleavage of the drug conjugates of the present invention in the extracellular space allows the drug to diffuse to neighboring cells and kill them. It is also envisaged that dying cells will liberate cleavage agents (e.g. cysteine or glutathione) that will activate more of the drug from the drug conjugate resulting in self-amplification of the toxic effects.

The present inventors have been able to isolate, affinity mature and study specific recombinant antibodies towards MMP-1, MMP-2 and MMP-3 which are highly overexpressed in cancer tissues. While all three MMPs were strongly expressed in diseased specimens, in particular MMP-3 could be efficiently reached by antibodies, as demonstrated in quantitative biodistribution studies [Pfaffen, S. et al (2010) Eur. J. Nucl. Med. Mol. Imaging, 37, 1559]. For this reason we MMP-3 is considered to be a particularly suitable target compared to other MMPs of the modified extracellular matrix at the site of disease.

Other targets of vascular localisation may include, but are not limited to, ROBO4, EndoPDI, DEL1, GP34, STC1, GA733, TEM1, TEM5, TEM7, TEM8, DELTA4, Endomucin, Annexin A1, Annexin A8, Ephrin A7, Myeloperoxidase, Nucleolin, Transferrin receptor, Vitamin D binding protein, VEGF receptor 1, VEGF receptor 2, TIE2, aminopeptidase-N, endoglin (CD105), CD66, CD44, CD13, Neuropilin-1, Endoglin, HES, PSMA and ASPP1, as described in Nature Reviews. Cancer (2005), vol. 5, 436-446. Prostate specific membrane antigen (PSMA) is considered to be especially suitable.

Other targets of vascular localisation of the cytotoxic compound may include, but are not limited to fibroblast growth factor receptor-1, CD31, tumour lymphatic endothelium, and alpha V beta 3 integrin, periostin, putative G-protein coupled receptor 42, solute carrier family 2, facilitated glucose transporter member 1, versican core protein, CEACAM3, Fibromodulin, Peroxidasin homolog, probable G-protein coupled receptor 37, protein sidekick-1, alpha1A-voltage-dependent calcium channel, EMILIN2 protein, down syndrome critical region protein 8, probable G-protein coupled receptor 113, ANXA4 protein, uromodulin-like 1, m(16) scavenger receptor class F member 2, Sushi domain-containing protein 2, tumour protein, translationally controlled 1, putative G-protein coupled receptor Q8TDUO, hypothetical protein DKFZp686K0275, transmembrane protein TMEM55A, hypothetical protein Q8WYY4, family with sequence similarity 116, member A, UPF0240 protein C6orf66, CDNA FLJ45811 fis, clone NT2RP7014778, hypothetical protein DKFZp77901248, beta-ureidopropionase, hypothetical protein DKFZp434F1919, cysteine-rich with EGF-like domain protein 2, UPF0378 family protein KIAA0100, potassium voltage-gated channel subfamily H member 1.

Other especially suitable targets for binding moieties of the invention include splice isoforms of fibronectin and splice isoforms of tenascin

Fibronectin (FN) is a glycoprotein and is widely expressed in a variety of normal tissues and body fluids. It is a component of the extracellular matrix (ECM), and plays a role in many biological processes, including cellular adhesion, cellular migration, haemostasis, thrombosis, wound healing, tissue differentiation and oncogenic transformation. Different FN isoforms are generated by alternative splicing of three regions (ED-A, ED-B, IIICS) of the primary transcript FN pre-mRNA, a process that is modulated by cytokines and extracellular pH. Fibronectin contains two type-III globular extra-domains which may undergo alternative splicing: ED-A and ED-B.

The ED-B domain of fibronectin corresponds to a sequence of 91 aminoacids identical in mouse, rat and human. Because it specifically accumulates around neo-vascular structures (Castellani et al. (1994). Int. J. Cancer 59, 612-618) it represents a target for molecular intervention with non internalizing binding members.

The ED-A domain of fibronectin is a 90 amino acid sequence. The ED-As of mouse fibronectin and human fibronectin are 96.7% identical (only 3 amino acids differ between the two 90 amino acid sequences). It is located between domain 11 and 12 of FN (Borsi et al., 1987, J. Cell Biol., 104, 595-600). ED-A is mainly absent in the plasma form of FN but is abundant during embryogenesis, tissue remodeling, fibrosis, cardiac transplantation and solid tumour growth. Just like for EDB, because it specifically accumulates around neo-vascular structures it represents a target for molecular intervention with non internalizing binding members.

Tenascin-C is a large hexameric glycoprotein of the extracellular matrix which modulates cellular adhesion. It is involved in processes such as cell proliferation and cell migration and is associated with changes in tissue architecture as occurring during morphogenesis and embryogenesis as well as under tumorigenesis or angiogenesis.

Several isoforms of tenascin-C can be generated as a result of alternative splicing which may lead to the inclusion of (multiple) domains in the central part of this protein, ranging from domain A1 to domain D [Borsi L et al. Int J Cancer 1992; 52:688-692, Carnemolla B et al. Eur J Biochem 1992; 205:561-567]. It had previously been assumed that domains A1-D could be inserted or omitted “in block” in the tenascin-C molecule by a mechanism of alternative spicing, leading to “tenascin-C large” and “tenascin-C small” molecules [Borsi L et al. J Biol Chem 1995; 270:6243-6245]. A strong over-expression of the large isoform of tenascin-C has been reported for a number of tumors [Borsi 1992 supra], and two monoclonal antibodies specific for domains A1 and D, respectively, have been extensively characterized in the clinic [Riva P et al. Int J Cancer 1992; 51:7-13, Riva P et al. Cancer Res 1995; 55:5952s-5956s, Paganelli G et al. Eur J Nucl Med 1994; 21:314-321, Reardon D A et al. J Clin Oncol 2002; 20:1389-1397, Bigner D D et al. J Clin Oncol 1998; 16:2202-2212.

However, it has recently become clear that a more complex regulation of the alternative splicing mechanism takes place, leading to an increased molecular heterogeneity among the large isoforms of tenascin-C. For example, it has been reported that the extra domain C of tenascin-C displays a more restricted pattern of expression compared with the other alternatively spliced domains of tenascin-C [Carnemolla B et al. Am J Pathol 1999; 154:1345-1352], with a predominantly perivascular staining as depicted with immunohistochemistry. The C domain of tenascin-C is undetectable in most normal adult tissues, but is over-expressed in high-grade astrocytomas [Carnemolla B et al. Am J Pathol 1999; 154:1345-1352] and other tumor types. Further support for the heterogeneity between large tenascin-C isoforms comes from transcriptional analyses, which confirmed that large tenascin-C transcripts feature a heterogeneous composition [Katenkamp K et al. J Pathol 2004; 203:771-779]. An additional level of complexity is provided by the presence or absence of post-translational modifications (e.g. glycosylation), which may modify certain epitopes on the surface of individual protein domains and make them unavailable to a specific molecular recognition in vitro or in vivo to specific monoclonal antibodies.

Binding Moiety

In certain embodiments, the binding moiety is a low molecular weight binding moiety. Thus, the binding moiety is preferably not an antibody or an antibody fragment. Suitably, the molecular weight of the binding moiety is less than about 8,000, preferably less than about 3000, most preferably less than about 1000. In embodiments, the binding moiety (ligand) is a peptide. In other embodiments, the binding moiety (ligand) is not a peptide. The possibility to step away from antibodies and to use small organic molecule as ligands allows those molecules to have complexity with is amenable to chemical synthesis. For example, the conjugates of the invention may comprise two or more binding moieties each linked to the drug through the cleavable linker whereby each of the binding moieties can separately bind to the target protein to provide improved binding.

The binding moiety may be based on a compound that is known to bind strongly to the targets of interest, for example a matrix metalloproteinase inhibitor. Alternatively, the binding moiety may be identified by one or more known screening methods for identifying compounds that bind selectively to the target protein of interest.

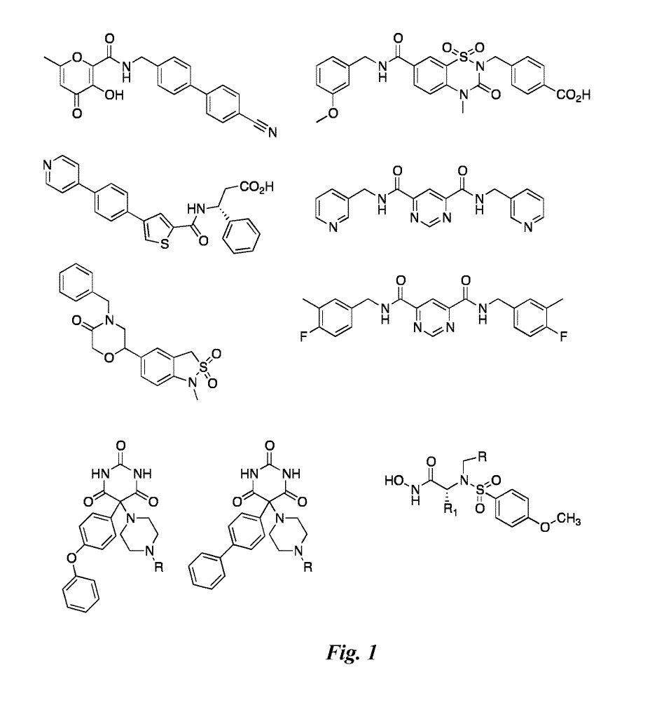

For example, the structures of MMP inhibitors shown in FIG. 1 are reported in literature and could form suitable ligands for targeting MMPs. The first six inhibitors are reported in Pirard, B. (2007) Drug Discov. Today, 12, 640. Two further inhibitors are based on pyrimidine-2,4,6-triones (or barbiturates). The R group indicates either aliphatic or aromatic substituents. [Schrigten, D. et al (2012) J. Med. Chem., 55, 223]. Finally, an inhibitor is shown based on hydroxamates, in which R can bear both aliphatic and aromatic chains, as well as triazole moieties, and the R₁ group can comprise a series of substituents including natural and unnatural amino acids side chains [Hugenberg, V. et al (2012) J. Med. Chem., 55, 4714].

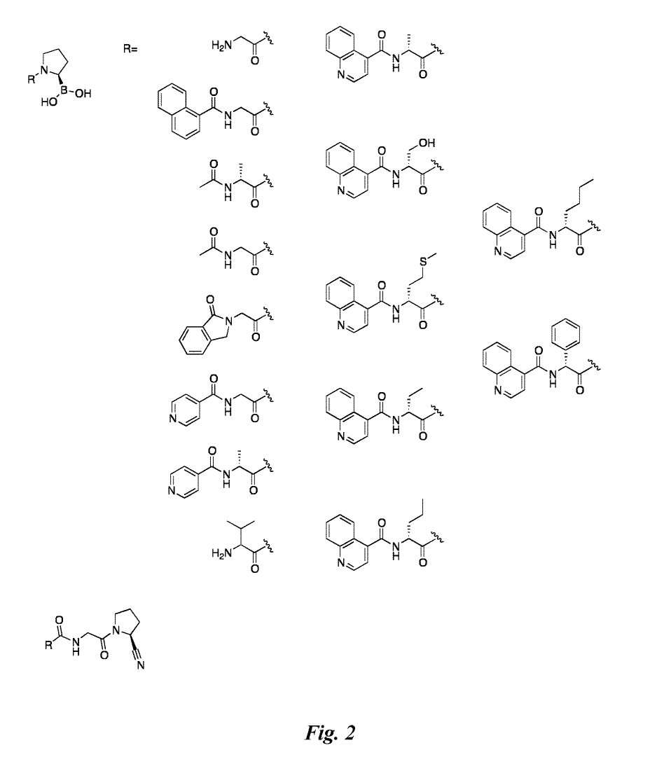

Reported structures of FAP inhibitors that could form the binding moiety of the conjugates of the present invention are shown in FIG. 2. The list includes a class of compounds based on a cyanopyrrolidine scaffold, in which the R residue could be a quinolone derivative. [Jasen, K. et al ACS Med Chem. Lett., 4, 491].

Finally there are reports with good membrane antigens (e.g. PSMA, Hillier, S. M., et al (2013) J. Nucl. Med, 54, 1369), that show good accumulation at tumors. In a particular example a class of PSMA ligands with affinity constants comprised between 1-10 nM, show selective tumor uptake in quantitative biodistribution studies with values up to 10% ID/g which are stable through 24 h. This last example is a clear demonstration that small organic molecules have great potential for selective in vivo targeting and accumulation.

In embodiments, the binding moiety B may be a univalent binding moiety or a multivalent binding moiety, for example a bivalent binding moiety. The term “univalent binding moiety” refers to a binding moiety comprising a single ligand for binding to the target entity. The term “multivalent binding moiety” refers to a binding moiety having two or more binding ligands (which may be the same or different) for binding to the target entity. The two or more binding ligands are separated by suitable spacer groups on the multivalent binding moieties. The use of multivalent binding moieties can provide enhanced binding of the binding moiety to the target.

Improved variants of the above ligands, or new ligands for binding selectively to target proteins of interest can be found by screening methods using modern medicinal chemistry technologies. In particular, suitable ligands can be found by screening DNA-encoded chemical libraries for example as described in WO2009077173 and by R E. Kleiner et al. in Chemical Society Reviews 40 5707-5717 (2011), L. Mannocci et al. in Chemical Communications 47, 12747-12753 (2011) and S. Brenner et al. in Proceedings of the National Academy of Sciences of the USA 89 5381-5383 (1992).

In other embodiments, the binding moiety comprises or consists essentially of an antibody or an antibody fragment. As discussed above, the term “antibody” describes an immunoglobulin whether natural or partly or wholly synthetically produced. The term also covers any polypeptide or protein having a binding domain which is, or is substantially homologous to, an antibody binding domain. Examples of antibodies are the immunoglobulin isotypes and their isotypic subclasses; fragments which comprise an antigen binding domain such as Fab, scFv, Fv, dAb, Fd; SIP and diabodies. It is possible to take monoclonal and other antibodies and use techniques of recombinant DNA technology to produce other antibodies or chimeric molecules which retain the specificity of the original antibody. Such techniques may involve introducing DNA encoding the immunoglobulin variable region, or the complementarity determining regions (CDRs), of an antibody to the constant regions, or constant regions plus framework regions, of a different immunoglobulin. See, for instance, EP-A-184187, GB 2188638A or EP-A-239400. A hybridoma or other cell producing an antibody may be subject to genetic mutation or other changes, which may or may not alter the binding specificity of antibodies produced.

As antibodies can be modified in a number of ways, the term “antibody” should be construed as covering any specific binding member or substance having a binding domain with the required specificity. Thus, this term covers antibody fragments, derivatives, functional equivalents and homologues of antibodies, including any polypeptide comprising an immunoglobulin binding domain, whether natural or wholly or partially synthetic. Chimeric molecules comprising an immunoglobulin binding domain, or equivalent, fused to another polypeptide are therefore included. Cloning and expression of chimeric antibodies are described in EP-A-0120694 and EP-A-0125023.

It has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward, E. S. et al., Nature 341, 544-546 (1989)) which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al, Science, 242, 423-426, 1988; Huston et al, PNAS USA, 85, 5879-5883, 1988); (viii) bispecific single chain Fv dimers (PCT/US92/09965) and (ix) “diabodies”, multivalent or multispecific fragments constructed by gene fusion (WO94/13804; P. Holliger et al, Proc. Natl. Acad. Sci. USA 90 6444-6448, 1993). Fv, scFv or diabody molecules may be stabilized by the incorporation of disulphide bridges linking the VH and VL domains (Y. Reiter et al. Nature Biotech 14 1239-1245 1996). Minibodies comprising an scFv joined to a CH3 domain may also be made (S. Hu et al, Cancer Res. 56 3055-3061 1996).

Diabodies are multimers of polypeptides, each polypeptide comprising a first domain comprising a binding region of an immunoglobulin light chain and a second domain comprising a binding region of an immunoglobulin heavy chain, the two domains being linked (e.g. by a peptide linker) but unable to associate with each other to form an antigen binding site: antigen binding sites are formed by the association of the first domain of one polypeptide within the multimer with the second domain of another polypeptide within the multimer (WO94/13804).

Where bispecific antibodies are to be used, these may be conventional bispecific antibodies, which can be manufactured in a variety of ways (Holliger, P. and Winter G. Current Opinion Biotechnol. 4, 446-449 (1993)), e.g. prepared chemically or from hybrid hybridomas, or may be any of the bispecific antibody fragments mentioned above. Diabodies and scFv can be constructed without an Fc region, using only variable domains, potentially reducing the effects of anti-idiotypic reaction.

Bispecific diabodies, as opposed to bispecific whole antibodies, may also be particularly useful because they can be readily constructed and expressed in E. coli. Diabodies (and many other polypeptides such as antibody fragments) of appropriate binding specificities can be readily selected using phage display (WO94/13804) from libraries. If one arm of the diabody is to be kept constant, for instance, with a specificity directed against antigen X, then a library can be made where the other arm is varied and an antibody of appropriate specificity selected.

Bispecific whole antibodies may be made by knobs-into-holes engineering (J. B. B. Ridgeway et al., Protein Eng. 9 616-621, 1996).

With reference to the discussion of Fibronectin ED-A and ED-B domains and the Tenascin-C A1 domain above, the present inventors have identified the antibodies that are specific binding partners for these entities. These antibodies or appropriate fragments thereof can form a specific binding moiety in the conjugates of the present invention.

The VH of the anti-EDB L19 antibody corresponds to SEQ. ID n° 1

SEQ. ID no 1
EVQLLESGGGLVQPGGSLRLSCAASGFTFSSFSMSWVRQAPGKGLEW
VSSISGSSGTTYYADSVKGRFTISRDNSKNTLYLQMNSLRAEDTAVY
YCAKPFPYFDYWGQGTLVTVSS

The VL of L19 antibody corresponds to SEQ. ID n° 2

SEQ. ID. no 2
EIVLTQSPGTLSLSPGERATLSCRASQSVSSSFLAWYQQKPGQAPRL
LIYYASSRATGIPDRFSGSGSGTDFTLTISRLEPEDFAVYYCQQTGR
IPPTFGQGTKVEIK

Anti-EDA Antibody

The VH of the anti-EDA F8 antibody corresponds to SEQ. ID n° 3

SEQ. ID. no 3
EVQLLESGGGLVQPGGSLRLSCAASGFTFSLFTMSWVRQAPGKGLEW
VSAISGSGGSTYYADSVKGRFTISRDNSKNTLYLQMNSLRAEDTAVY
YCAKSTHLYLFDYWGQGTLVTVSS

The VL of the anti-EDA F8 antibody corresponds to SEQ. ID n° 4

SEQ. ID no 4
EIVLTQSPGTLSLSPGERATLSCRASQSVSMPFLAWYQQKPGQAPRL
LIYGASSRATGIPDRFSGSGSGTDFTLTISRLEPEDFAVYYCQQMRG
RPPTFGQGTKVEIK

Anti-TNA1 Antibody

The VH of the anti-TNA1 F16 antibody corresponds to SEQ. ID n° 5

SEQ. ID. no 5
EVQLLESGGGLVQPGGSLRLSCAASGFTFSRYGMSWVRQAPGKGLEW
VSAISGSGGSTYYADSVKGRFTISRDNSKNTLYLQMNSLRAEDTAVY
YCAKAHNAFDYWGQGTLVTVSR

The VL of the anti-TNA1 F16 antibody corresponds to SEQ. ID n° 6

SEQ. ID. no 6
SSELTQDPAVSVALGQTVRITCQGDSLRSYYASWYQQKPGQAPVLVI
YGKNNRPSGIPDRFSGSSSGNTASLTITGAQAEDEADYYCNSSVYTM
PPVVFGGGTKLTVL

Some antibodies that preferred for use in the present invention include the human monoclonal antibodies F8 (specific to the alternatively spliced EDA domain of fibronectin—see Int. J Cancer (2008), 122, 2405-2413; WO2008/120101); L19 (specific to the alternatively spliced EDB domain of fibronectin—see ATCC Patent Deposit PTA-9529 and the sequence which is set forth herein); and F16 (specific to the alternatively spliced A1 domain of tenascin-C—see Clin. Cancer Res. (2006) 12, 3200-3208; WO2010/078916).

In embodiments, the binding moiety B may be a univalent binding moiety or a multivalent binding moiety, for example a bivalent binding moiety. The term “univalent binding moiety” refers to a binding moiety comprising a single ligand for binding to the target entity. The term “multivalent binding moiety” refers to a binding moiety having two or more binding ligands (which may be the same or different) for binding to the target entity. Suitably, the binding moiety is bivalent. The two or more binding ligands are separated by suitable spacer groups on the multivalent binding moieties. The use of multivalent binding moieties can provide enhanced binding of the binding moiety to the target.

Linker

The linker attaches the binding moiety to the drug moiety e.g. through one or more covalent bond(s). The linker may be a bifunctional or a multifunctional moiety which can be used to link one or more drug moieties and/or binder moieties to form the drug conjugate of the invention.

Those cytotoxic payloads should stably remain attached to their ligand while in circulation, but should be released when the conjugate reaches the site of disease.

Release mechanisms are identical to those specific to antibodies linked to cytotoxic payloads. Indeed the nature of the ligand is independent on that respect. Therefore we can envisage pH-dependent [Leamon, C. P. et al (2006) Bioconjugate Chem., 17, 1226; Casi, G. et al (2012) J. Am. Chem. Soc., 134, 5887], reductive [Bernardes, G. J. et al (2012) Angew. Chem. Int Ed. Engl. 51. 941; Yang, J. et al (2006) Proc. Natl. Acad. Sci. USA, 103, 13872] and enzymatic release [Doronina S. O. et al (2008) Bioconjugate Chem, 19, 1960; Sutherland, M. S. K. (2006) J. Biol. Chem, 281, 10540]. In a specific setting, when functional groups are present on either the ligand or payloads (e.g. thiols, alcohols) a linkerless connection can be established thus releasing intact payloads, which simplifies substantially pharmacokinetic analysis.

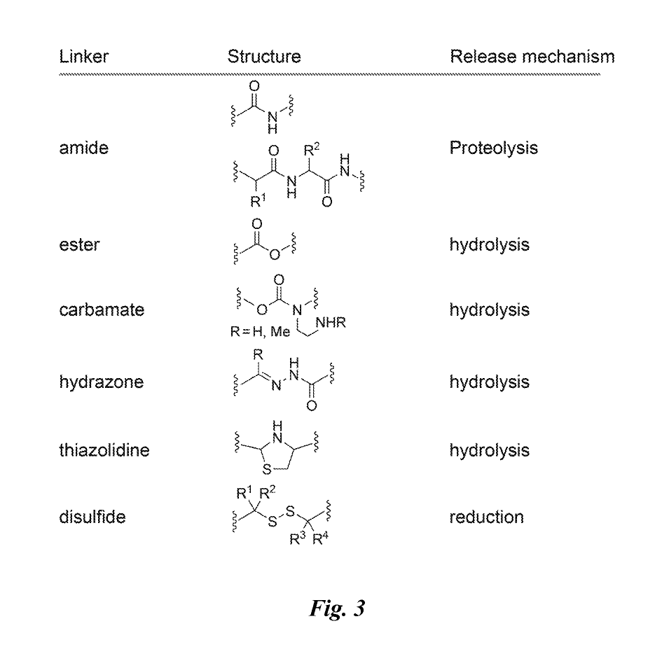

A non-exhaustive list of linkers is shown in FIG. 3, wherein the substituents R and Rⁿ shown in the formulas may suitably be independently selected from H, halogen, substituted or unsubstituted (hetero)alkyl, (hetero)alkenyl, (hetero)alkynyl, (hetero)aryl, (hetero)arylalkyl, (hetero)cycloalkyl, (hetero)cycloalkylaryl, heterocyclylalkyl, a peptide, an oligosaccharide or a steroid group. Suitably R and Rⁿ are independently selected from H, or C1-C7 alkyl or heteroalkyl. More suitably, R and Rⁿ are independently selected from H, methyl or ethyl.

Suitably, the conjugate is stable to hydrolysis. That is to say, less than about 10% of the conjugate undergoes hydrolysis in PBS pH7.4 at 37° C. after 24 hours, as determined by HPLC.

Accordingly, the linker suitably comprises as its cleavable bond a disulfide linkage since these linkages are stable to hydrolysis, while giving suitable drug release kinetics at the target in vivo, and can provide traceless cleavage of drug moieties including a thiol group, such as DM1.

Suitably, the linker may be polar or charged in order to improve water solubility of the conjugate. For example, the linker may comprise from about 1 to about 20, suitably from about 2 to about 10, residues of one or more known water-soluble oligomers such as peptides, oligosaccharides, glycosaminoglycans, polyacrylic acid or salts thereof, polyethylene glycol, polyhydroxyethyl (meth) acrylates, polysulfonates, etc. Suitably, the linker may comprise a polar or charged peptide moiety comprising e.g. from 2 to 10 amino acid residues. Amino acids may refer to any natural or non-natural amino acid. The peptide linker suitably includes a free thiol group, preferably a N-terminal cysteine, for forming the said cleavable disulfide linkage with a thiol group on the drug moiety. A suitable peptide linker of this type is -Cys-Asp-Arg-Asp-.

Suitably, the linker is linked to the ligand through a 1,2,3-triazole ring formed by 1,3-cycloaddition of alkyne and azide. The drug and binding moieties are suitably linked to the 3 and 5 positions of the triazole ring. The triazole ring may optionally be substituted at the 4 position. For example, the conjugates according to the present invention may have the following formula:

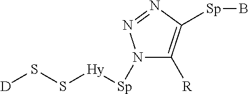

wherein: Hy is a hydrophilic moiety for improving the solubility of the conjugate, for example a hydrophilic oligomer as defined above such as a peptide group as defined above. S—S represents the cleavable disulfide bond between the drug moiety D and the linker. Suitably, the disulfide bond is formed between a —SH group on the linker, for example the —SH group of a cysteine residue (preferably terminal cysteine) of the peptide and a —SH group present in the active form of the drug D, for example the terminal —SH group of DM1. In this way, reductive cleavage of the disulfide bond in vivo results in traceless release of the drug in its active form.

Sp are spacer groups, which may be independently selected from optionally substituted straight or branched or cyclic C1-C6 alkylene or alkenylene, optionally including one or more carbonyl carbons or ether or thioether O or S atoms or amine N atoms in the chain. The first Sp group is suitably linked to the peptide residue by a terminal carbonyl forming an amide linkage with the terminal amino group of the peptide.

The triazole is optionally substituted at the 4 position by group R, whereby group R is selected from H or any of the substituent groups defined herein, or R is substituted or unsubstituted (hetero)alkyl, (hetero)alkenyl, (hetero)alkynyl, (hetero)aryl, (hetero)arylalkyl, (hetero)cycloalkyl, (hetero)cycloalkylaryl, heterocyclylalkyl, a peptide, an oligosaccharide or a steroid group. Suitably R is selected from H, halogen, halomethyl, or C1-C7 alkyl or heteroalkyl. More suitably, R is selected from H, methyl or ethyl, and most suitably R is H.

In these and other embodiments, the linker comprises a peptide unit that is specifically tailored so that it will be selectively enzymatically cleaved from the drug moiety by one or more proteases on the cell surface or the extracellular regions of the target tissue. The amino acid residue chain length of the peptide unit suitably ranges from that of a single amino acid to about eight amino acid residues. Numerous specific cleavable peptide sequences suitable for use in the present invention can be designed and optimized in their selectivity for enzymatic cleavage by a particular tumor-associated enzyme e.g. a protease. Cleavable peptides for use in the present invention include those which are optimized toward the proteases MMP-1, 2 or 3, or cathepsin B, C or D. Especially suitable are peptides containing the sequence Val-Cit, which are cleavable by Cathepsin B. Cathepsin B is a ubiquitous cysteine protease. It is an intracellular enzyme, except in pathological conditions, such as metastatic tumors or rheumatoid arthritis. Therefore, non-internalizing conjugates of the present invention produced with cathepsin B-cleavable linkers are stable in circulation until activated in pathological tissue.

In these embodiment, the linker moiety suitably further comprises, adjacent to the peptide sequence, a “self-immolative” linker portion. The self-immolative linkers are also known as electronic cascade linkers. These linkers undergo elimination and fragmentation upon enzymatic cleavage of the peptide to release the drug in active, preferably free form. The conjugate is stable extracellularly in the absence of an enzyme capable of cleaving the linker.

However, upon exposure to a suitable enzyme, the linker is cleaved initiating a spontaneous self-immolative reaction resulting in the cleavage of the bond covalently linking the self-immolative moiety to the drug, to thereby effect release of the drug in its underivatized or pharmacologically active form. In these embodiments, the self-immolative linker is coupled to the ligand moiety through an enzymatically cleavable peptide sequence that provides a substrate for an enzyme to cleave the amide bond to initiate the self-immolative reaction. Suitably, the drug moiety is connected to the self-immolative moiety of the linker via a chemically reactive functional group pending from the drug such as a primary or secondary amine, hydroxyl, sulfhydryl or carboxyl group.

Examples of self-immolative linkers are PABC or PAB (para-aminobenzyloxycarbonyl), attaching the drug moiety to the ligand in the conjugate (Carl et al (1981) J. Med. Chem. 24: 479-480; Chakravarty et al (1983) J. Med. Chem. 26: 638-644). The amide bond linking the carboxy terminus of a peptide unit and the para-aminobenzyl of PAB may be a substrate and cleavable by certain proteases. The aromatic amine becomes electron-donating and initiates an electronic cascade that leads to the expulsion of the leaving group, which releases the free drug after elimination of carbon dioxide (de Groot, et al (2001) Journal of Organic Chemistry 66 (26): 8815-8830). Further self-immolating linkers are described in WO2005/082023.

In these embodiments, the linker suitably further comprises a spacer unit linked to the binding moiety, for example via an amide, amine or thioether bond. The spacer unit is of a length that enables e.g. the cleavable peptide sequence to be contacted by the cleaving enzyme (e. g. cathepsin B) and suitably also the hydrolysis of the amide bond coupling the cleavable peptide to the self-immolative moiety X. Spacer units may for example comprise a divalent radical such as alkylene, arylene, a heteroarylene, repeating units of alkyloxy (e.g. polyethylenoxy, PEG, polymethyleneoxy) and alkylamino (e.g. polyethyleneamino), or diacid ester and amides including succinate, succinamide, diglycolate, malonate, and caproamide.

In yet other embodiments, the linker comprises a glucuronyl group that is cleavable by glucoronidase present on the cell surface or the extracellular region of the target tissue. It has been shown that lysosomal beta-glucuronidase is liberated extracellularly in high local concentrations in necrotic areas in human cancers, and that this provides a route to targeted chemotherapy (Bosslet, K. et al. Cancer Res. 58, 1195-1201 (1998)).

Drug

In one embodiment, the drug is a cytotoxic agent that inhibits or prevents the function of cells and/or causes destruction of cells. Examples of cytotoxic agents include radioactive isotopes, chemotherapeutic agents, and toxins such as small molecule toxins or enzymatically active toxins of bacterial, fungal, plant or animal origin, including synthetic analogues and derivatives thereof. The cytotoxic agent may be selected from the group consisting of an auristatin, a DNA minor groove binding agent, a DNA minor groove alkylating agent, an enediyne, a lexitropsin, a duocarmycin, a taxane, a puromycin, a dolastatin, a maytansinoid and a vinca alkaloid or a combination of two or more thereof.

In one embodiment the drug is a chemotherapeutic agent selected from the group consisting of a topoisomerase inhibitor, an alkylating agent (eg. nitrogen mustards; ethylenimes; alkylsulfonates; triazenes; piperazines; and nitrosureas), an antimetabolite (eg mercaptopurine, thioguanine, 5-fluorouracil), an antibiotics (eg. anthracyclines, dactinomycin, bleomycin, adriamycin, mithramycin, dactinomycin) a mitotic disrupter (eg. plant alkaloids—such as vincristine and/or microtubule antagonists—such as paclitaxel), a DNA intercalating agent (eg carboplatin and/or cisplatin), a DNA synthesis inhibitor, a DNA-RNA transcription regulator, an enzyme inhibitor, a gene regulator, a hormone response modifier, a hypoxia-selective cytotoxin (eg. tirapazamine), an epidermal growth factor inhibitor, an anti-vascular agent (eg. xanthenone 5,6-dimethylxanthenone-4-acetic acid), a radiation-activated prodrug (eg. nitroarylmethyl quaternary (NMQ) salts) or a bioreductive drug or a combination of two or more thereof.

The chemotherapeutic agent may selected from the group consisting of Erlotinib (TARCEVA®), Bortezomib (VELCADE®), Fulvestrant (FASLODEX®), Sutent (SU11248), Letrozole (FEMARA®), Imatinib mesylate (GLEEVEC®), PTK787/ZK 222584, Oxaliplatin (Eloxatin®.), 5-FU (5-fluorouracil), Leucovorin, Rapamycin (Sirolimus, RAPAMUNE®.), Lapatinib (GSK572016), Lonafarnib (SCH 66336), Sorafenib (BAY43-9006), and Gefitinib (IRESSA®.), AG1478, AG1571 (SU 5271; Sugen) or a combination of two or more thereof.

The chemotherapeutic agent may be an alkylating agent—such as thiotepa, CYTOXAN® and/or cyclosphosphamide; an alkyl sulfonate—such as busulfan, improsulfan and/or piposulfan; an aziridine—such as benzodopa, carboquone, meturedopa and/or uredopa; ethylenimines and/or methylamelamines—such as altretamine, triethylenemelamine, triethylenepbosphoramide, triethylenethiophosphoramide and/or trimethylomelamine; acetogenin—such as bullatacin and/or bullatacinone; camptothecin; bryostatin; callystatin; cryptophycins; dolastatin; duocarmycin; eleutherobin; pancratistatin; sarcodictyin; spongistatin; nitrogen mustards—such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide and/or uracil mustard; nitrosureas—such as carmustine, chlorozotocin, fotemustine, lomustine, nimustine, and/or ranimnustine; dynemicin; bisphosphonates—such as clodronate; an esperamicin; a neocarzinostatin chromophore; aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN®. doxorubicin—such as morpholino-doxorubicin, cyanomorpholino-doxorubicin, 2-pyrrolino-doxorubicin and/or deoxydoxorubicin, epirubicin, esorubicin, idarubicin, marcellomycin, mitomycins—such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites—such as methotrexate and 5-fluorouracil (5-FU); folic acid analogues—such as denopterin, methotrexate, pteropterin, trimetrexate; purine analogues—such as fludarabine, 6-mercaptopurine, thiamiprine, thioguanine; pyrimidine analogues—such as ancitabine, azacitidine, 6-azauridine, carmofur, cytarabine, dideoxyuridine, doxifluridine, enocitabine, floxuridine; androgens—such as calusterone, dromostanolone propionate, epitiostanol, mepitiostane, testolactone; anti-adrenals—such as aminoglutethimide, mitotane, trilostane; folic acid replenisher—such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; macrocyclic depsipeptides such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; razoxane; rhizoxin; sizofiran; spirogermanium; tenuazonic acid; triaziquone; 2,2′,2″-trichlorotriethylamine; trichothecenes—such as verracurin A, roridin A and/or anguidine; urethan; vindesine; dacarbazine; mannomustine; mitobronitol; mitolactol; pipobroman; gacytosine; arabinoside; cyclophosphamide; thiotepa; taxoids—such as TAXOL®. paclitaxel, abraxane, and/or TAXOTERE®, doxetaxel; chloranbucil; GEMZAR®. gemcitabine; 6-thioguanine; mercaptopurine; methotrexate; platinum analogues—such as cisplatin and carboplatin; vinblastine; platinum; etoposide; ifosfamide; mitoxantrone; vincristine; NAVELBINE®, vinorelbine; novantrone; teniposide; edatrexate; daunomycin; aminopterin; xeloda; ibandronate; topoisomerase inhibitor RFS 2000; difluoromethylomithine (DMFO); retinoids—such as retinoic acid; capecitabine; and pharmaceutically acceptable salts, acids, derivatives or combinations of two or more of any of the above.

The drug may be a tubulin disruptor including but are not limited to: taxanes—such as paclitaxel and docetaxel, vinca alkaloids, discodermolide, epothilones A and B, desoxyepothilone, cryptophycins, curacin A, combretastatin A-4-phosphate, BMS 247550, BMS 184476, BMS 188791; LEP, RPR 109881A, EPO 906, TXD 258, ZD 6126, vinflunine, LU 103793, dolastatin 10, E7010, T138067 and T900607, colchicine, phenstatin, chalcones, indanocine, T138067, oncocidin, vincristine, vinblastine, vinorelbine, vinflunine, halichondrin B, isohomohalichondrin B, ER-86526, pironetin, spongistatin 1, spiket P, cryptophycin 1, LU103793 (cematodin or cemadotin), rhizoxin, sarcodictyin, eleutherobin, laulilamide, VP-16 and D-24851 and pharmaceutically acceptable salts, acids, derivatives or combinations of two or more of any of the above.

The drug may be a DNA intercalator including but are not limited to: acridines, actinomycins, anthracyclines, benzothiopyranoindazoles, pixantrone, crisnatol, brostallicin, CI-958, doxorubicin (adriamycin), actinomycin D, daunorubicin (daunomycin), bleomycin, idarubicin, mitoxantrone, cyclophosphamide, melphalan, mitomycin C, bizelesin, etoposide, mitoxantrone, SN-38, carboplatin, cis-platin, actinomycin D, amsacrine, DACA, pyrazoloacridine, irinotecan and topotecan and pharmaceutically acceptable salts, acids, derivatives or combinations of two or more of any of the above.

The drug may be an anti-hormonal agent that acts to regulate or inhibit hormone action on tumours—such as anti-estrogens and selective estrogen receptor modulators, including, but not limited to, tamoxifen, raloxifene, droloxifene, 4-hydroxytamoxifen, trioxifene, keoxifene, LY117018, onapristone, and/or fareston toremifene and pharmaceutically acceptable salts, acids, derivatives or combinations of two or more of any of the above. The drug may be an aromatase inhibitor that inhibits the enzyme aromatase, which regulates estrogen production in the adrenal glands—such as, for example, 4(5)-imidazoles, aminoglutethimide, megestrol acetate, AROMASIN®. exemestane, formestanie, fadrozole, RIVISOR®. vorozole, FEMARA®. letrozole, and ARIMIDEX® and/or anastrozole and pharmaceutically acceptable salts, acids, derivatives or combinations of two or more of any of the above.

The drug may be an anti-androgens—such as flutamide, nilutamide, bicalutamide, leuprolide, goserelin and/or troxacitabine and pharmaceutically acceptable salts, acids, derivatives or combinations of two or more of any of the above.

The drug may be a protein kinase inhibitor, a lipid kinase inhibitor or an anti-angiogenic agent.

The drug could also be a cytokine (e.g., an interleukin, a member of the TNF superfamily, or an interferon).

In a preferred embodiment, the drug is a maytansinoid, in particular DM1, or a tubulin disruptor.

The drug may be used in unmodified or modified form. Combinations of drugs in which some are unmodified and some are modified may be used. For example, the drug may be chemically modified. One form of chemical modification is the derivatisation of a carbonyl group—such as an aldehyde. According to one embodiment, the drug is modified to allow the incorporation of the linker.

In a preferred embodiment, the drug is a maytansinoid, in particular mertansine (DM1), or a tubulin disruptor. Preferably, the drug in its active form comprises a thiol group, whereby a cleavable disulfide bond may be formed through the sulfur of the thiol group to bond the drug to the linker moiety in the conjugates of the invention.

The drug may be used in unmodified or modified form. Combinations of drugs in which some are unmodified and some are modified may be used. For example, the drug may be chemically modified. One form of chemical modification is the derivatisation of a carbonyl group—such as an aldehyde.

According to one embodiment, the drug is modified to allow the incorporation of the linker. For example, a drug comprising a hydroxyl group may be converted to the corresponding 2-ethanethiol carbonate or 2-ethanethiol carbamate thereby introducing thiol groups for disulphide linkage.

Drug Conjugates

The drug moiety of the drug conjugate of the present invention may not be cleaved from the linker until the drug conjugate binds to its target cell or tissue.

In one embodiment, the drug conjugates described herein are not internalised into a cell. Accordingly, the linker that is used in the drug conjugate should be stable enough compared to the rate of blood clearance of the compound but labile enough compared to the residence time of the compound at the target site. From these considerations, a half-life of the linker in the region of about 1 hour to about 50 hours—such as about 10 to about 50 hours, about 20 to about 50 hours, about 30 hours to about 50 hours, about 30 hours to about 45 hours, about 35 hours to 45 hours, about 35 hours to 40 hours, or about 37 hours—may be acceptable, especially when vascular tissues or cells are targeted. Still longer half-lives may be appropriate for embodiments in which the drug conjugate is activated in vivo by subsequent administration of an exogenous cleavage agent as discussed further below. For example, half lives greater than about 40 hours, suitably greater than about 50 hours, greater than about 60 hours , greater than about 72 hours, or greater than about 96 hours.

Advantageously therefore, the drug conjugates described herein may have improved lability and/or stability in vitro and/or in vivo which makes them particularly suitable for controlled drug release, especially at vascular tissues, cells and tumours.

Suitably, the drug conjugate shows a high affinity for tumors when administered systemically. Suitably, a tumor-to-blood concentration ratio of at least about 5:1, for example at least about 10:1 is achieved 1 hour after injection of 3 nM of the conjugate into nude mice having subcutaneous SKRC52 tumors.

Suitably, the drug conjugate inhibits, retards or prevents growth of a tumour when administered in a therapeutically effective amount. For example, the compound when administered to balb/c nu/nu mice having subcutaneous SKRC52 tumors daily for seven consecutive days at a maximum dose selected to cause less than 5% weight loss after 10 days causes a greater reduction in tumor growth than an equimolar dose of the same drug in active, untargeted form.

Treatment

The drug conjugates described herein may be used to treat disease. The treatment may be therapeutic and/or prophylactic treatment, with the aim being to prevent, reduce or stop an undesired physiological change or disorder. The treatment may prolong survival as compared to expected survival if not receiving treatment.

The disease that is treated by the drug conjugate may be any disease that might benefit from treatment. This includes chronic and acute disorders or diseases including those pathological conditions which predispose to the disorder. One particular disease that is applicable to treatment by the present invention is neoplastic disease such as cancer that can be treated via the targeted destruction of the established tumour vasculature. Non-limiting examples of cancers that may be treated include benign and malignant tumours; leukemia and lymphoid malignancies, including breast, ovarian, stomach, endometrial, salivary gland, lung, kidney, colon, thyroid, pancreatic, prostate or bladder cancer. The disease may be a neuronal, glial, astrocytal, hypothalamic or other glandular, macrophagal, epithelial, stromal and blastocoelic disease; or inflammatory, angiogenic or an immunologic disease. An exemplary disease is a solid, malignant tumour.

The term “cancer” and “cancerous” is used in its broadest sense as meaning the physiological condition in mammals that is typically characterized by unregulated cell growth. A tumour comprises one or more cancerous cells. Examples of cancer include, but are not limited to, carcinoma, lymphoma, blastoma, sarcoma, and leukemia or lymphoid malignancies. Further examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, gastrointestinal stromal tumour (GIST), pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

For the prevention or treatment of disease, the dosage of an ADC will depend on an array of different factors—such as the type of disease to be treated, the severity and course of the disease, whether the molecule is administered for preventive or therapeutic purposes, previous therapy, the patient's clinical history and response to the protein, and the discretion of the attending physician.

The molecule may be administered to the patient at one time or over a series of treatments. Depending on the type and severity of the disease, between about 1 ug/kg to 15 mg/kg of drug may be used as an initial candidate dosage for administration to the patient, whether, for example, by one or more separate administrations, or by continuous infusion. A typical daily dosage might range from about 1 ug/kg to 100 mg/kg or more. An exemplary dosage of drug may be in the range of about 0.1 to about 10 mg/kg of patient weight.

When treating cancer, the therapeutically effect that is observed may be a reduction in the number of cancer cells; a reduction in tumour size; inhibition or retardation of cancer cell infiltration into peripheral organs; inhibition of tumour growth; and/or relief of one or more of the symptoms associated with the cancer.

In animal models, efficacy may be assessed by physical measurements of the tumour during the treatment, and/or by determining partial and complete remission of the cancer. For cancer therapy, efficacy can, for example, be measured by assessing the time to disease progression (TTP) and/or determining the response rate (RR).

Pharmaceutical Compositions

The drug conjugates described herein may be in the form of pharmaceutical compositions which may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilisers, dyes and even flavouring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition may be formulated to be administered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be administered by a number of routes.

If the agent is to be administered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions may be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or the pharmaceutical compositions can be injected parenterally, for example, intravenously, intramuscularly or subcutaneously. For parenteral administration, the compositions may be best used in the form of a sterile aqueous solution which may contain other substances, for example, enough salts or monosaccharides to make the solution isotonic with blood. For buccal or sublingual administration the compositions may be administered in the form of tablets or lozenges which can be formulated in a conventional manner.

The drug conjugate of the present invention may be administered in the form of a pharmaceutically acceptable or active salt. Pharmaceutically-acceptable salts are well known to those skilled in the art, and for example, include those mentioned by Berge et al, in J. Pharm. Sci., 66, 1-19 (1977). Salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts.

The routes for administration (delivery) may include, but are not limited to, one or more of oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, vaginal, epidural, sublingual.

Typically, a physician will determine the actual dosage which will be most suitable for an individual subject. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

The formulations may be packaged in unit-dose or multi-dose containers, for example sealed ampoules and vials, and may be stored in a freeze-dried (lyophilized) condition requiring only the addition of the sterile liquid carrier, for example water, for administration. Extemporaneous injection solutions and suspensions are prepared from sterile powders, granules and tablets of the kind previously described. Exemplary unit dosage formulations contain a daily dose or unit daily sub-dose, or an appropriate fraction thereof, of the active ingredient.

Combination Therapy

A drug conjugate of the present invention may be combined in a pharmaceutical combination formulation, or dosing regimen as combination therapy, with a second compound having therapeutic properties. The second compound of the pharmaceutical combination formulation or dosing regimen preferably has complementary activities to the drug conjugate of the combination such that they do not adversely affect each other.

The second compound may be selected from the group consisting of a protein, antibody, antigen-binding fragment thereof, a drug, a toxin, an enzyme, a nuclease, a hormone, an immunomodulator, an antisense oligonucleotide, an siRNA, a boron compound, a photoactive agent, a dye and a radioisotope or a combination of two or more thereof.

The combination therapy may be administered as a simultaneous or sequential regimen. When administered sequentially, the combination may be administered in two or more administrations. The combined administration includes coadministration, using separate formulations or a single pharmaceutical formulation, and consecutive administration in either order, wherein there is a time period while both (or all) active agents simultaneously exert their biological activities.

As noted above, the drug conjugates of the invention achieve optimal tumor:organ ratios some time after administration, when the drug conjugate has had the opportunity to localize at the site of the disease, while clearing from blood and healthy organs. Thus, it would be desirable to provide controlled release of the toxic payload from the drug conjugate at a controlled time interval after administration. This can be achieved by administering an effective amount of a cleavage agent for cleaving the linker L at a later time point following drug conjugate administration, in order to trigger an efficient release of the drug payload when suitable tumor:blood and tumor:organ ratios have been achieved. The time interval between administration of the drug conjugate and administration of the cleavage agent may, for example, be from about 10 minutes to about 12 hours, suitably from about 30 minutes to about 6 hours, more suitably from about 1 hour to about 2 hours.

Thus, the combination products according to the invention include a product comprising a compound of Formula (I) as defined above and a cleavage agent for cleaving the cleavable linker L, as a combined preparation for sequential administration in the treatment of cancer.

Suitably, either: (a) linker L comprises a disulphide bond and the cleavage agent comprises a reducing agent such as cysteine, N-acetylcysteine, ordithiothreitol; or (b) linker L comprises an amide linkage and the cleavage agent comprises a hydrolase such as a protease; or (c) linker L comprises an ester linkage and the cleavage agent comprises a hydrolase such as an esterase.

The cleavage agent is administered in an amount effective to achieve the desired release of the toxic payload from the drug conjugate in vivo. For example, between about 1 μg/kg to 15 mg/kg of drug may be used as an initial candidate dosage for administration to the patient, whether, for example, by one or more separate administrations, or by continuous infusion. An exemplary dosage of cleavage agent may be in the range of about 0.1 to about 10 mg/kg of patient weight.

The above products for combined administration and methods of treatment by sequential administration of drug conjugate and cleavage agent are also applicable to antibody-drug conjugates as to conjugates in which the ligand is a low molecular weight entity. Thus, combination products and methods in which the drug conjugate is an antibody-drug conjugate (ADC) are encompassed within these aspects of the invention.

General Techniques

The practice of the present invention employs, unless otherwise indicated, conventional methods of chemistry, biochemistry, molecular biology, cell biology, genetics, immunology and pharmacology, known to those of skill of the art. Such techniques are explained fully in the literature. See, e.g., Gennaro, A. R., ed. (1990) Remington's Pharmaceutical Sciences, 18th ed., Mack Publishing Co.; Hardman, J. G., Limbird, L. E., and Gilman, A. G., eds. (2001) The Pharmacological Basis of Therapeutics, 10th ed., McGraw-Hill Co.; Colowick, S. et al., eds., Methods In Enzymology, Academic Press, Inc.; Weir, D. M., and Blackwell, C. C., eds. (1986) Handbook of Experimental Immunology, Vols. I-IV, Blackwell Scientific Publications; Maniatis, T. et al., eds. (1989) Molecular Cloning: A Laboratory Manual, 2nd edition, Vols. I-III, Cold Spring Harbor Laboratory Press; Ausubel, F. M. et al., eds. (1999) Short Protocols in Molecular Biology, 4th edition, John Wiley & Sons; Ream et al., eds. (1998) Molecular Biology Techniques: An Intensive Laboratory Course, Academic Press; Newton, C. R., and Graham, A., eds. (1997) PCR (Introduction to Biotechniques Series), 2nd ed., Springer Verlag.

All publications cited herein are incorporated herein by reference in their entirety for the purpose of describing and disclosing the methodologies, reagents, and tools reported in the publications that might be used in connection with the invention.

Chemical Synthesis

The compounds described herein may be prepared by chemical synthesis techniques. It will be apparent to those skilled in the art that sensitive functional groups may need to be protected and deprotected during synthesis of a compound. This may be achieved by conventional techniques, for example as described in “Protective Groups in Organic Synthesis” by T W Greene and P G M Wuts, John Wiley and Sons Inc. (1991), and by P. J. Kocienski, in “Protecting Groups”, Georg Thieme Verlag (1994).

It is possible during some of the reactions that any stereocentres present could, under certain conditions, be epimerised, for example if a base is used in a reaction with a substrate having an optical centre comprising a base-sensitive group. It should be possible to circumvent potential problems such as this by choice of reaction sequence, conditions, reagents, protection/deprotection regimes, etc. as is well-known in the art.

The compounds and salts of the invention may be separated and purified by conventional methods.

The invention will now be further described by way of Examples, which are meant to serve to assist one of ordinary skill in the art in carrying out the invention and are not intended in any way to limit the scope of the invention.

### EXAMPLES

Compounds

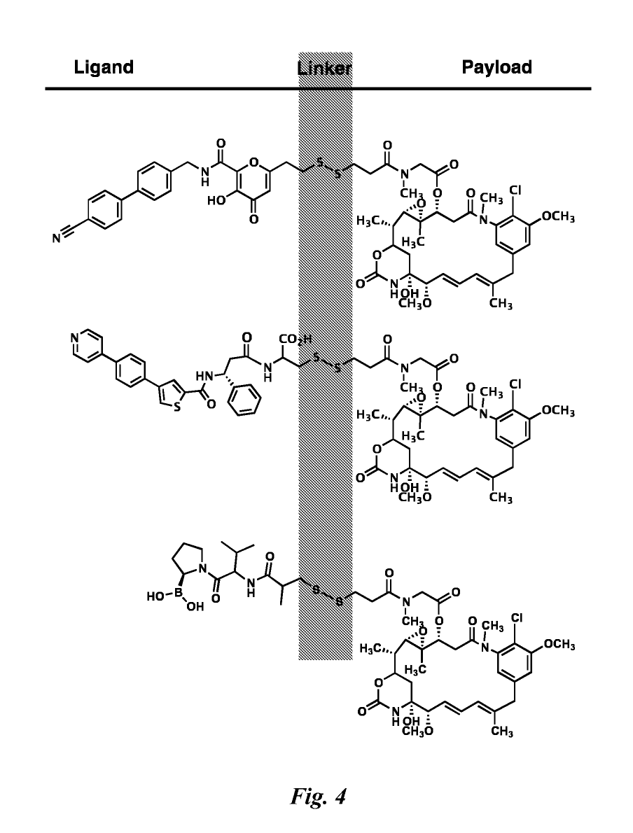

FIG. 4 shows representative drug conjugates according to the invention. The drug moiety in each case is mertansine (DM1). DM1 has a terminal thiol group, which forms one half of the cleavable disulfide linker bond in these conjugates. The binder moieties in these examples comprise two different MMP inhibitor moieties as described above, derivatized with thiol-containing terminal groups for forming the disulfide link. The binding moiety of the third compound is another ligand for a tumor ECM protein.

Evaluation of the Antitumor Activity and Toxicity of Auristatin (MMAE) Conjugated with F8 Antibody Binding Moiety and Cathepsin B-Cleavable Peptide Linker.

129Sv female mice were injected subcutaneously with 10⁷ F9 murine teratocarcinoma cells. Mice underwent treatment for 5 consecutive days starting from day 11 after tumor transplantation. Mice received equimolar amounts of:
(i) the cytotoxic drug MMAE as free drug (MMAE 0.325 mg/kg),
(ii) unconjugated F8 antibody in SIP format (10 mg/kg)
(iii) the F8 antibody in SIP format (10 mg/kg) conjugated to MMAE via a Maleimido Caproyl Valine Citrulline Para-Amino Benzyl (MC-VC-PAB) linker (L)
(i) the F8 antibody in IgG format (20 mg/kg) conjugated to MMAE via a Maleimido Caproyl Valine Citrulline Para-Amino Benzyl (MC-VC-PAB) linker (L)
(ii) PBS (negative control)

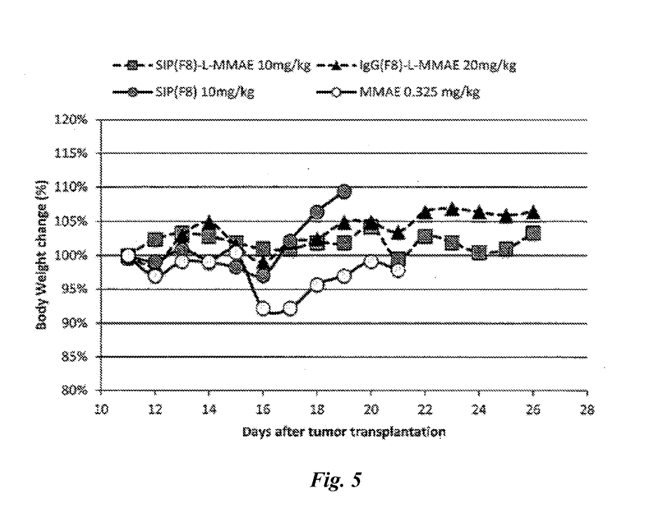

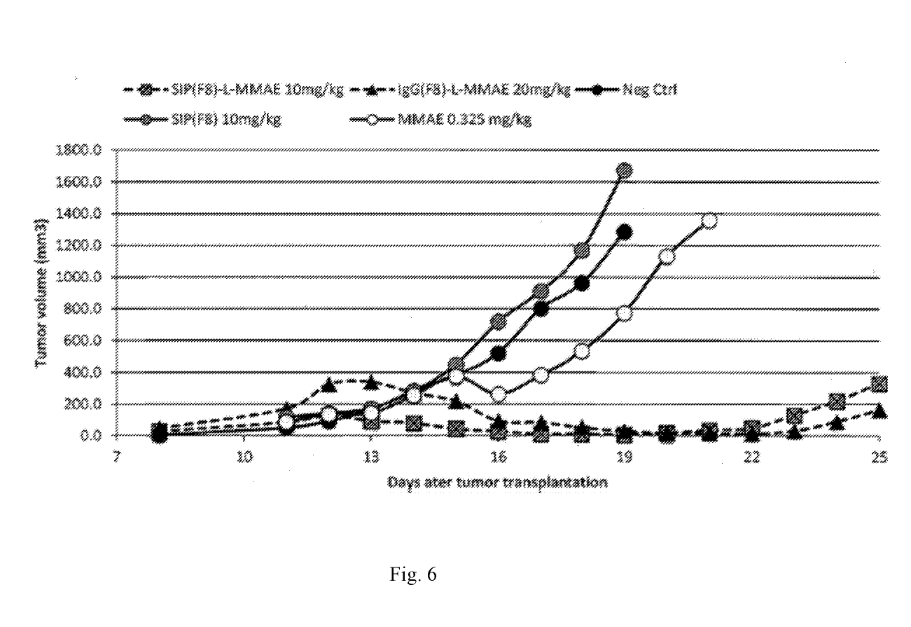

Efficacy and toxicity data are shown, respectively, in FIGS. 5 and 6. It can be seen that the conjugates substantially reduced tumor volume from day 15 onwards, relative to the controls and unconjugated MMAE, with reduced toxicity relative to unconjugated MMAE.

Evaluation of the Antitumor Activity and Toxicity of Auristatin (MMAE) Conjugated with F16 Antibody Binding Moiety and Cathepsin B-Cleavable Peptide Linker.

Balb/c nu/nu female mice were injected subcutaneously with 4×10⁶ U87 human glioblastoma cell line.

Mice underwent treatment for 5 consecutive days starting from day 19 after tumor transplantation. The mice received:
(i) unconjugated F16 antibody in SIP format (10 mg/kg)
(ii) the F16 antibody in SIP format (10 mg/kg) conjugated to MMAE via a Maleimido CaproylValine Citrulline Para-Amino Benzyl (MC-VC-PAB) linker (L)
(iii) the F16 antibody in SIP format (2 mg/kg) conjugated to MMAE via a Maleimido CaproylValine Citrulline Para-Amino Benzyl (MC-VC-PAB) linker (L)
(iv) the F16 antibody in SIP format (2 mg/kg) conjugated to MMAE via
(v) Herceptin™ antibody in SIP format conjugated to DM1 via a disulphide linker (2 mg/kg)

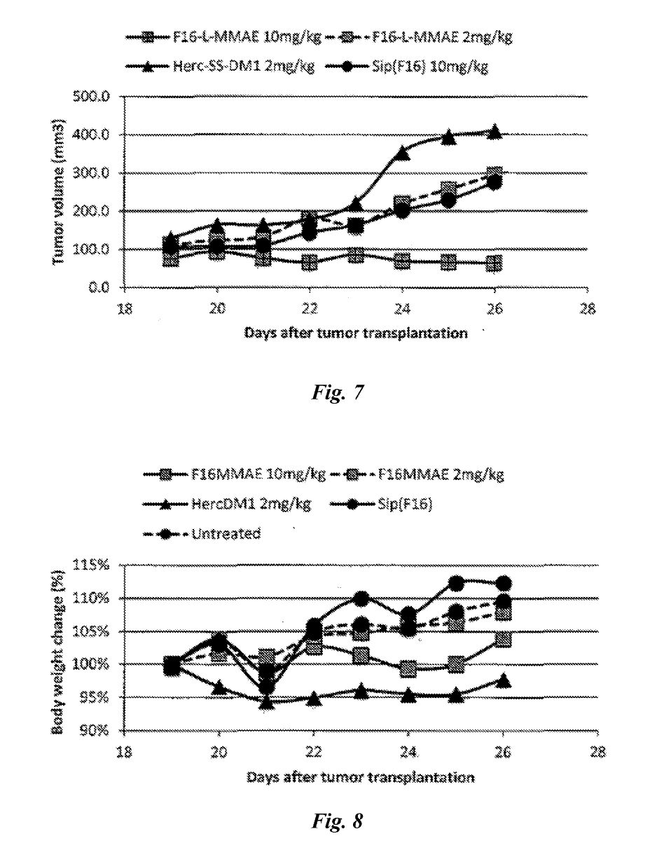

As further reference body weights of untreated, healthy mice as also plotted. Efficacy and toxicity data are shown, respectively, in FIGS. 7 and 8. It can be seen that the F16 conjugates at 10 mg/kg substantially inhibited tumor growth, with reduced toxicity relative to the Herceptin conjugate.

All patent documents and other references cited herein are expressly incorporated herein by reference.

The above embodiments of the invention have been described for the purpose of illustration only. Many other embodiments falling within the scope of the accompanying claims will be apparent to the skilled reader.

1. A targeted therapeutic agent comprising a compound of formula:
B-L-D
wherein:
B is a non-internalizing antibody or an antibody fragment specific for a cancer associated extracellular matrix protein which is fibronectin having alternatively spliced EDA sub-domains;
D is a cytotoxic drug moiety; and
L is a linker group that undergoes cleavage in vivo for releasing said drug moiety in an active form.

2. A targeted therapeutic agent according to claim 1, wherein said antibody or antibody fragment is multivalent, having two or more ligands for binding to a target entity.

3. A targeted therapeutic agent according to claim 1, wherein said antibody or antibody fragment comprises a non internalizing antibody, such as a non-internalizing IgG or scFv or Fab or SIP or diabody.

4. A targeted therapeutic agent according to claim 3, wherein the non internalizing antibody is specific for the ED-A domain of fibronectin.

5. A targeted therapeutic agent according to claim 1, wherein said cytotoxic drug moiety is a tubulin disruptor, for example a maytansinoid, in particular mertansine (DM1).

6. A targeted therapeutic agent according to claim 1, wherein said cytotoxic drug moiety in active form comprises a thiol group for forming a disulfide bond to said linker in said compound.

7. A targeted therapeutic agent according to claim 1, wherein said linker comprises a disulfide bond that undergoes cleavage in vivo to release said drug moiety.

8. A targeted therapeutic agent according to claim 1, wherein said linker comprises a polar or charged moiety for improving water solubility of the conjugate.

9. A targeted therapeutic agent according to claim 8, wherein said polar or charged moiety is an oligomer comprising from 1 to about 20 monomers selected from the group consisting of natural and non-natural amino acids, saccharides, (meth)acrylic acid and salts thereof, hydroxyethyl (meth)acrylate, and ethylene glycol.

10. A targeted therapeutic agent according to claim 9, wherein said polar or charged moiety is an oligomer comprising from 2 to about 10 monomers.

11. A targeted therapeutic agent according to claim 1, wherein said linker comprises a cysteine group for linking to said drug moiety through a disulfide bond.

12. A targeted therapeutic agent according to claim 1, wherein said linker L comprises a 1,2,3-triazole ring, wherein said drug moiety and binder moiety are linked to positions 1 and 4 of the triazole ring and the 5 position of the triazole ring is also optionally substituted.

13. A targeted therapeutic agent according to claim 1, wherein the linker comprises a peptide that is cleavable by a protease that is present in the extracellular matrix of a tumor or that is released after tumor cell death.

14. A targeted therapeutic agent according to claim 13 wherein the peptide is cleavable by MMP-1, MMP-2, MMP-3, or Cathepsin A, B, or C.

15. A targeted therapeutic agent according to claim 14 wherein the linker comprises valine-citrulline and is cleavable by cathepsin B.

16. A targeted therapeutic agent according to claim 13 wherein the peptide-containing linker further comprises a self-immolating spacer.

17. A targeted therapeutic agent according to claim 1, wherein the linker contains a glucuronide moiety, that is cleavable by glucuronidases.

18. A targeted therapeutic agent comprising a compound of formula:
B-L-D
wherein:
B is a non-internalizing antibody or an antibody fragment specific for a cancer associated extracellular matrix protein which is tenascin-C having the extra domain A1;
D is a cytotoxic drug moiety; and
L is a linker group that undergoes cleavage in vivo for releasing said drug moiety in an active form.

19. A targeted therapeutic agent according to claim 18, wherein said antibody or antibody fragment is multivalent, having two or more ligands for binding to a target entity.

20. A targeted therapeutic agent according to claim 18, wherein said antibody or antibody fragment comprises a non-internalizing antibody.

21. A targeted therapeutic agent according to claim 20, wherein the non internalizing antibody is specific for the domain A1 of tenascin.

22. A targeted therapeutic agent according to claim 18, wherein said cytotoxic drug moiety is a tubulin disruptor.

23. A targeted therapeutic agent according to claim 18, wherein said cytotoxic drug moiety in active form comprises a thiol group for forming a disulfide bond to said linker in said compound.

24. A targeted therapeutic agent according to claim 18, wherein said linker comprises a disulfide bond that undergoes cleavage in vivo to release said drug moiety.

25. A targeted therapeutic agent according to claim 18, wherein said linker comprises a polar or charged moiety for improving water solubility of the conjugate.

26. A targeted therapeutic agent according to claim 25, wherein said polar or charged moiety is an oligomer comprising from 1 to about 20 monomers selected from the group consisting of natural and non-natural amino acids, saccharides, (meth)acrylic acid and salts thereof, hydroxyethyl (meth)acrylate, and ethylene glycol.

27. A targeted therapeutic agent according to claim 26, wherein said polar or charged moiety is an oligomer comprising from 2 to about 10 monomers.

28. A targeted therapeutic agent according to claim 18, wherein said linker comprises a cysteine group for linking to said drug moiety through a disulfide bond.

29. A targeted therapeutic agent according to claim 18, wherein said linker L comprises a 1,2,3-triazole ring, wherein said drug moiety and binder moiety are linked to positions 1 and 4 of the triazole ring and the 5 position of the triazole ring is also optionally substituted.

30. A targeted therapeutic agent according to claim 18, wherein the linker comprises a peptide that is cleavable by a protease that is present in the extracellular matrix of a tumor or that is released after tumor cell death.

31. A targeted therapeutic agent according to claim 30 wherein the peptide is cleavable by MMP-1, MMP-2, MMP-3, or Cathepsin A, B, or C.

32. A targeted therapeutic agent according to claim 31 wherein the linker comprises valine-citrulline and is cleavable by cathepsin B.

33. A targeted therapeutic agent according to claim 30 wherein the peptide-containing linker further comprises a self-immolating spacer.

34. A targeted therapeutic agent according to claim 18, wherein the linker contains a glucuronide moiety, that is cleavable by glucuronidases.

35. A targeted therapeutic agent having the general formula: 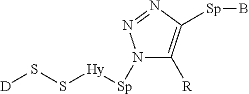
wherein:
B is a non-internalizing antibody or an antibody fragment specific for a cancer associated extracellular matrix protein which is tenascin-C having the extra domain A1;
D is a cytotoxic drug moiety;
the intervening structure is a Linker;
Hy is a hydrophilic moiety for improving the solubility of the agent;
S—S represents a cleavable disulfide bond between the drug moiety D and the linker;
Sp are spacer groups, which may be independently selected from optionally substituted straight or branched or cyclic C1-C6 alkylene or alkenylene, optionally including one or more carbonyl carbons or ether or thioether O or S atoms or amine N atoms in the chain; and
R is selected from H, halogen, carboxylate, substituted or unsubstituted (hetero)alkyl, (hetero)alkenyl, (hetero)alkynyl, (hetero)aryl, (hetero)arylalkyl, (hetero)cycloalkyl, (hetero)cycloalkylaryl, heterocyclylalkyl, a peptide, an oligosaccharide or a steroid group.

36. A targeted therapeutic agent having the general formula: 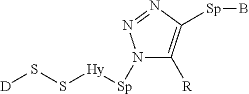
wherein:
B is a non-internalizing antibody or an antibody fragment specific for a cancer associated extracellular matrix protein which is fibronectin having alternatively spliced EDA sub-domains;
D is a cytotoxic drug moiety;
the intervening structure is a linker;
Hy is a hydrophilic moiety for improving the solubility of the agent;
S—S represents a cleavable disulfide bond between the drug moiety D and the linker;
Sp are spacer groups, which may be independently selected from optionally substituted straight or branched or cyclic C1-C6 alkylene or alkenylene, optionally including one or more carbonyl carbons or ether or thioether O or S atoms or amine N atoms in the chain; and
R is selected from H, halogen, carboxylate, substituted or unsubstituted (hetero)alkyl, (hetero)alkenyl, (hetero)alkynyl, (hetero)aryl, (hetero)arylalkyl, (hetero)cycloalkyl, (hetero)cycloalkylaryl, heterocyclylalkyl, a peptide, an oligosaccharide or a steroid group.

36. A targeted therapeutic agent comprising a compound of formula:
B-L-D
wherein:
B is a non-internalizing antibody or an antibody fragment specific for a cancer associated extracellular matrix protein comprising one of the structures selected from the group consisting of: 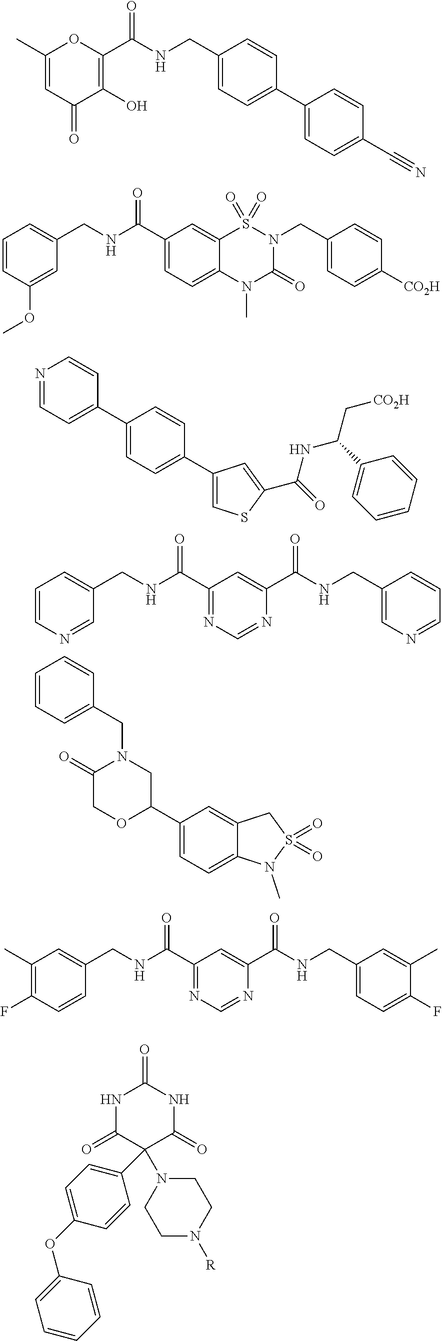 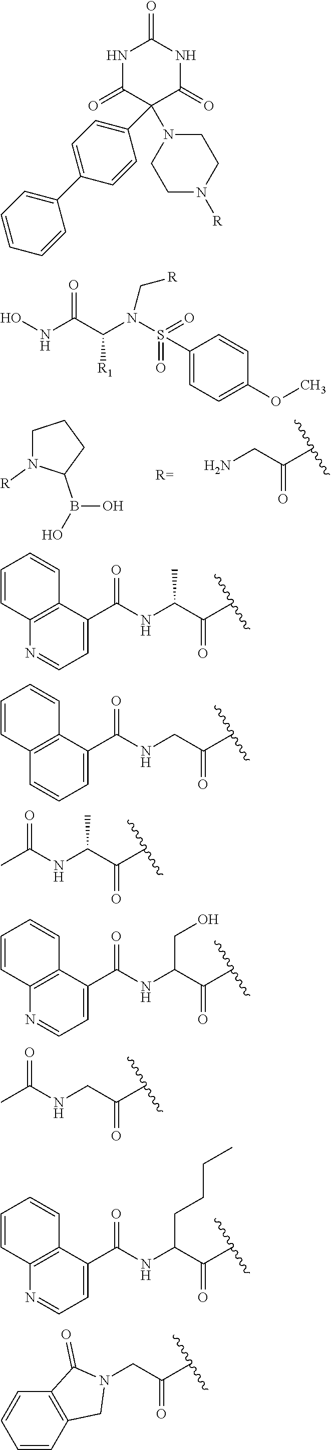 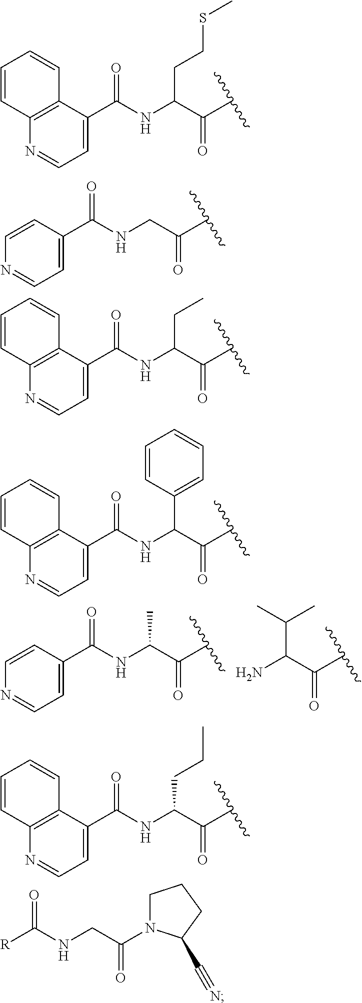
D is a cytotoxic drug moiety; and
L is a linker group that undergoes cleavage in vivo for releasing said drug moiety in an active form.

37. A targeted therapeutic agent comprising a compound selected from the group consisting of:

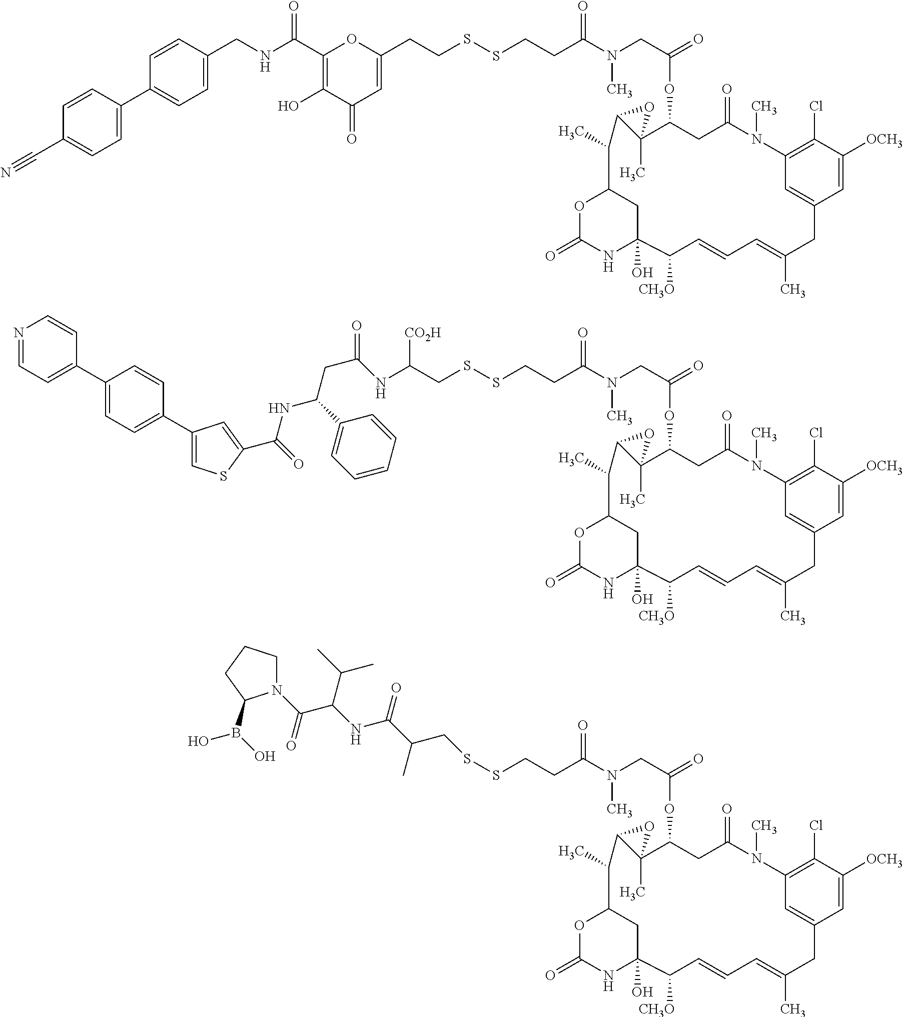

38. A targeted therapeutic agent comprising a compound of formula:
B-L-D
wherein:
B is a non-internalizing antibody or an antibody fragment specific for a cancer associated extracellular matrix protein; and
L is a linker group that undergoes cleavage in vivo for releasing said drug moiety in an active form;
wherein said compound when administered to balb/c nu/nu mice having subcutaneous SKRC52 tumors daily for five consecutive days at a maximum dose selected to cause less than 5% weight loss after 10 days causes a greater reduction in tumor growth than an equimolar dose of the same drug in active, untargeted form.

39. A method for treating a neoplastic disease in a subject, comprising administering to a subject in need thereof a targeted therapeutic agent comprising a compound of formula:
B-L-D
wherein:
B is a non-internalizing antibody or an antibody fragment specific for a cancer associated extracellular matrix protein;
D is a cytotoxic drug moiety; and
L is a linker group that undergoes cleavage in vivo for releasing said drug moiety in an active form.

40. A method according to claim 39, for the treatment of a solid tumor.

41. A method according to claim 39, for the treatment of renal cell carcinoma.

42. A product comprising the compound of claim 1 and a cleavage agent for cleaving said cleavable linker L, as a combined preparation for sequential administration in the treatment of cancer.

43. A product according to claim 42, wherein either: (a) linker L comprises a disulphide bond and the cleavage agent comprises a reducing agent such as cysteine, N-acetylcysteine, ordithiothreitol; or (b) linker L comprises an amide linkage and said cleavage agent comprises a hydrolase such as a protease; or (c) linker L comprises an ester linkage and said cleavage agent comprises a hydrolase such as an esterase.

44. The targeted therapeutic agent of claim 20, wherein said non-internalizing antibody is selected from the group consisting of non-internalizing IgG, scFv, Fab, SIP, and diabody.

45. The targeted therapeutic agent of claim 22, wherein said tubulin disruptor is selected from the group consisting of a maytansinoid and mertansine (DM1).


SEQUENCE LISTING (7 sequences)

SEQ ID NO: 1 - protein, 116 aa, Homo sapiens [sequence omitted]

SEQ ID NO: 2 - protein, 108 aa, Homo sapiens [sequence omitted]

SEQ ID NO: 3 - protein, 118 aa, Homo sapiens
EVQLLESGGGLVQPGGSLRLSCAASGFTFSLFTMSWVRQAPGKGLEWVSAISGSGGSTYYADSVKGRFTISRDNSKNTLYLQMNSLRAEDTAVYYCAKSTHLYLFDYWGQGTLVTVSS

SEQ ID NO: 4 - protein, 108 aa, Homo sapiens
EIVLTQSPGTLSLSPGERATLSCRASQSVSMPFLAWYQQKPGQAPRLLIYGASSRATGIPDRFSGSGSGTDFTLTISRLEPEDFAVYYCQQMRGRPPTFGQGTKVEIK

SEQ ID NO: 5 - protein, 116 aa, Homo sapiens
EVQLLESGGGLVQPGGSLRLSCAASGFTFSRYGMSWVRQAPGKGLEWVSAISGSGGSTYYADSVKGRFTISRDNSKNTLYLQMNSLRAEDTAVYYCAKAHNAFDYWGQGTLVTVSR

SEQ ID NO: 6 - protein, 108 aa, Homo sapiens
SSELTQDPAVSVALGQTVRITCQGDSLRSYYASWYQQKPGQAPVLVIYGKNNRPSGIPDRFSGSSSGNTASLTITGAQAEDEADYYCNSSVYTMPPVVFGGGTKLTVL

SEQ ID NO: 7 - protein, 4 aa, Artificial sequence
  Synthetic peptide linker
CDRD